(19) United States
(12) Patent Application Publication
(10) Pub. No.: US 2014/0017257 A1
(43) Pub. Date: Jan. 16, 2014

(54) IgY From Norovirus P Particles And Their Derivatives

(71) Applicants: Xi JIANG, Cincinnati, OH (US); Ying-Chun Dai, Guangdong (CN)

(72) Inventors: Xi JIANG, Cincinnati, OH (US); Ying-Chun Dai, Guangdong (CN)

(21) Appl. No.: 13/842,091

(22) Filed: Mar. 15, 2013

Related U.S. Application Data
(60) Provisional application No. 61/670,361, filed on Jul. 11, 2012.

(51) Int. Cl. C07K 16/02

(52) U.S. Cl. CPC C07K 16/02
USPC 424/159.1; 424/164.1

(57) ABSTRACT

A method for large-scale production of anti-NoV antibodies for use as a potential treatment for NoV disease using passive immunization. NoV-specific immunoglobulins (IgY) can be produced by immunizing chickens with NoV P particles. The birds continuously produced high titers of antibodies in their eggs for at least 3 months, in which NoV-specific antibody levels reached 4.7-9.2 mg/egg yolk. The egg yolk antibodies strongly reacted with NoV P particles by both ELISA and Western blot and blocked NoV virus-like particle (VLP) and P particle binding to the histo-blood group antigen (HBGA) receptors with a BT₅₀ of about 1:800. The chicken IgY remain stable at 70° C. for 30 min or treatment at pH 4 to 9 for 3 hr, demonstrating that chicken IgY provides large-scale production of anti-NoV antibodies for use in passive immunization against NoV infection.

### CROSS REFERENCE TO RELATED APPLICATIONS

This application claims the benefit of U.S. Provisional application 61/670,361, filed Jul. 11, 2012, the disclosure of which is incorporated by reference in its entirety.

### INTEREST/GOVERNMENT SUPPORT

This invention was supported under AI 055649, AI 37093, AI089634 and HD 13021 awarded by National Institutes of Health, and 2011-68003-30005 awarded by the USDA National Institute of Food and Agriculture. The government has certain rights in the invention.

### BACKGROUND OF THE INVENTION

Noroviruses (NoVs) are globally important pathogens, responsible for more than 90% of outbreaks of non-bacterial acute gastroenteritis. NoV outbreaks occur in a wide variety of settings including nursing homes, hospitals, day-care centers, cruise ships, restaurants, and catered events. Although NoV infection is normally mild and self-limited, severe cases have been observed in immunocompromised patients and the elderly. NoVs also result in over a million hospitalizations; with about 900,000 clinic visits and 200,000 deaths of children under 5 years of age in developing countries. Unfortunately, there are no vaccines or antivirals currently commercially available against NoVs.

NoVs are non-enveloped RNA viruses that contain a single-stranded, positive sense RNA genome. The genome is encompassed by a protein capsid that is formed by a single major structural protein, the capsid protein (VP1) and a minor structural protein (VP2). The VP1 capsid protein can be divided into two major domains, the N-terminal shell (S) domain and the C-terminal protrusion (P) domain. The P domain can form dimers, 12-mer small P particles, and 24-mer P-domain particles (“P particles”) as described in US Patent Publication US 2009/0280139, the disclosure of which is incorporated by reference in its entirety, when it is expressed in E. coli. While all three of these P complexes are immunogenic and recognize HBGAs, the 24-mer P particle is particularly useful as a candidate vaccine because of its high immunogenicity, stability, and low cost of production.

NoV P domain contains three surface loops that are useful for presentation of foreign antigens for immune enhancements. Insertion of one antigen into one loop result in 24 copies of the antigen on the P particle surfaces and up to 72 copies of the antigens on the P particles are expected to be developed if all three surfaces are used. Bacterial and viral antigens that have been successfully inserted on the P particles included the rotavirus spike protein VP8*, influenza virus M2e antigens, the measles surface protein, etc.

Passive immunization remains an effective strategy to prevent and treat infectious diseases. Oral administration of antibodies derived from mammalian serum has been previously described. However, the high cost of large-scale antibody production in mammals has limited its application. Passive immunization with monoclonal antibodies has also been shown to have lower levels of protection compared to polyclonal antibodies. The recently developed chicken IgY approach provides a useful alternative for large-scale production of polyclonal antibodies at a lower cost. Chicken IgYs are made in the blood and transferred to the egg yolk during embryo development. Since egg yolks are easily harvested, the IgY technology became a promising strategy to prevent and control infectious diseases, especially for gastrointestinal infections.

### SUMMARY OF THE INVENTION

The present invention provides for a method for producing NoV-specific IgY and IgY against P particles carrying other, variable surface antigens (including, but not limited to, the rotavirus (RV) VP8*) in the egg yolks of birds, typically chickens, immunized with NoV P particles and their derivatives, in large amounts and with high titer. The NoV-specific IgYs are stable at wide temperature and pH ranges.

The present invention also provides a method for passive immunization, comprising administering to a person having a compromised or weakened immunity system at least one dose of a composition comprising NoV-specific IgYs and IgY against other viral antigens, including but not limited to RV VP8* sufficient to treat, ameliorate, inhibit or prevent infection by NoV and RVs.

The anti-NoV and other anti-RV IgY of the present invention react strongly to NoV virus-like particles (VLPs) and P particles and RV VP8* in both ELISA and Western blot techniques, and were capable of blocking NoV-HBGA receptor interactions, which demonstrate that the anti-NoV and anti-RV IgY are useful for large-scale production for therapeutic use against NoVs and RVs.

The present invention relates to a method for treatment and prophylaxis of viral and bacterial infections, which is both safe and effective. This is achieved by using IgY directed against a virus or other pathogen, and in particular against Norovirus (NoV) and other virus, including rotavirus (RV), which has been obtained from the egg yolk of birds, that have been hyperimmunised with a viral protein particle antigen, and in particular a NoV P Particle and a NoV P particle with RV VP8* surface loop antigen or other surface loop antigens, to stimulate the production of immunoglobulines (IgY) against the antigens. The present invention also relates to a pharmaceutical product from eggs of birds containing immunoglobuline or a fraction thereof, for use in the passive prophylaxis or therapy of gastrointestinal infections in newborn infants and other persons having a compromised, immature or weakened immune system. The pharmaceutical IgY product can include a buffering agent.

### BRIEF DESCRIPTION OF THE DRAWINGS

FIG. 1 shows the kinetics of serum anti-NoV IgY response of chickens after immunization with NoV P particles.

FIG. 2 shows the kinetics of egg yolk anti-NoV IgY response of chickens after immunization with NoV P particles, including the amount of NoV-specific IgY per egg yolk (vertical columns, right-hand Y-axis).

FIG. 3 shows comparable reactivities of egg yolk IgY with NoV GII.4 (VA387) P particles (PP) and virus-like particles (VLP).

FIG. 4: Panel (a) shows Western blot analysis of anti-NoV chicken IgY against NoV P particle, where partially purified chicken egg yolk IgY was analyzed by SDS-PAGE; Lane 1: protein standards (Bio-RAD), lane 2: purified egg yolk IgY with indications of heavy and light chains. Panels (b) and (c) show the partially purified IgY (1:4000 dilution) interacts with GII.4 (VA387) P particles (panel c) following SDS-PAGE (panel b); Lane 1: protein standards, lane 2: NoV P particles.

FIG. 5 shows chicken IgY induced by NoV P particles blocked the binding of NoV P particles to HBGA receptors, wherein the partially purified IgY induced by NoV P particle (▪ symbol) in a wide range of dilutions can be used as a detection antibody to measure binding of NoV P particles to various saliva samples in saliva-based HBGA binding assays, versus control PBS (♦ symbol). The results were similar to those using guinea pig IgG induced by VLPs of strain VA387 (▴ symbol). Each data point represents an average value of six binding assays using six different type A salivas.

FIG. 6 shows the IgY induced by NoV P particles blocked binding of norovirus VLPs to HBGA receptors (♦ symbol), whereas IgY after immunization with PBS neither reacted with P particles (FIG. 5, ♦ symbol) nor showed detectable blocking (X symbol).

FIG. 7 shows the effect of temperature on the ability of IgY to block NoV P particle binding to HBGAs, using the GII.4 (VA387) P particles and a type A saliva sample in the blocking assay. The chicken IgY was treated with the indicated range of temperatures for 30 min before being tested in the blocking assay. A percentage (%) of residual blocking activity in comparison with an untreated sample is shown. Each assay was performed in triplicate and each data point represents the average of these assays.

FIG. 8 shows the effect of pH on the ability of IgY to block NoV P particle binding to HBGAs, using the GII.4 (VA387) P particles and a type A saliva sample in the blocking assay. The chicken IgY was treated with the indicated range of pHs for 3 hours before being tested in the blocking assay. A percentage (%) of residual blocking activity in comparison with an untreated sample is shown. Each assay was performed in triplicate and each data point represents the average of these assays.

### DETAILED DESCRIPTION OF THE INVENTION

IgY has been successfully used to treat and prevent infectious diseases in humans and domestic animals (Dias da Silva and Tambourgi, 2010; Vega et al., 2011). White leghorn chickens are easily raised and have high egg productivity. After immunization with a small dose of antigen, a chicken can continuously produce eggs containing antigen-specific antibodies in their yolks (Xu et al., 2011). A chicken usually lays 280 eggs/year and an egg yolk (12-15 ml) usually contains 150-200 mg IgY, of which 2 to 10% are specific antibodies (Nguyen et al., 2010). Thus, a chicken is referred as a small “factory” for antibody production.

NoVs and RVs are a leading cause of viral gastroenteritis, and remain hard to prevent and control due to their wide spread nature and lack of effective therapeutic and prophylactic methods. The methods and compositions of the present invention provide a method for passive immunization of high-risk populations, including young children, the elderly, and immunocompromised patients, whose immunity may be immature or weakened, by administering an effective dose(s) of anti-NoV or anti-RV IgY antibodies, to help limit or prevent persistent infections with NoVs and RVs

A continual increase of NoV-specific IgY in egg yolks was observed starting in the third week after the initial immunization. NoV specific antibody reached 4.7-9.2 mg/egg yolk between the fourth and the sixteenth weeks, which accounted for 2.5-5% of the total IgY. During this period, 340 eggs were collected from 4 immunized chickens and thus, a total of ˜2,400 mg specific anti-NoV IgY was obtained. Producing an equal amount of antibody would require 100-125 guinea pigs assuming that 15-20 ml sera can be collected from each animal. Strikingly, high levels of antigen-specific egg yolk IgY can be produced up to 2 years when the bird is continuously boosted every three months. Furthermore, collection of yolk antibody is non-invasive without affecting the immunized chickens. Finally, the NoV-specific IgY showed excellent reactivity at a wide range of temperature and pHs, an indication of highly stable IgY, important for clinical use.

Similar results are obtained within a NoV P particle with RV VP8* surface loop antigen*.

The NoV specific IgYs can block the binding of NoV VLPs/P particles to HBGAs. This is an important indication that the IgY is likely to neutralize, and therefore prevent, NoV infection and illness. A direct correlation between the ability of an antibody to block VLP-HBGA binding and protection against NoV infection and illness was observed in a recent NoV challenge study (Reeck et al., 2010). An additional study also showed that pre-existing HBGA blocking antibodies protected children from a GII.4 infection (Nurminen et al., 2011). Thus, the IgYs developed in accordance with the present invention are effective for prevention and treatment of NoV infection and illness using a passive immunization approach.

Similar high titer and yield of anti-RV antibodies have also been obtained by immunization of chickens with the P particle presenting other viral antigens, including the RV VP8* antigens.

### Preparation of P Particles and their Derivatives

P particles can be prepared as described in US Patent Publication US 2009/0280139, the disclosure of which is incorporated by reference in its entirety. By way of example, recombinant 24-mer P particles of strain VA387 (NoV P, GII.4, SEQ. ID. NO:1) can be expressed in E. coli. Purification of the glutathione S-transferase (GST)-NoV P⁻ fusion protein (SEQ. ID. NO:2) can be performed using resin of Glutathione Sepharose 4 Fast Flow (GE Healthcare life Sciences, NJ, USA) according to the manufacturer's instructions. GST (SEQ. ID. NO:3) can be removed from the target proteins by thrombin (GE Healthcare life Sciences, NJ, USA) cleavage either on beads or in solution (phosphate buffer saline, PBS, pH 7.4) at room temperature for 16 hr.

Similar procedures are used to prepare the P particles carrying at least one surface antigen, wherein the at least one surface antigen includes a bacterial antigen and a viral antigen. Non-limiting examples of bacterial antigens and viral antigens include the rotavirus spike protein VP8* (RV VP8*, SEQ. ID. NO:4), influenza virus M2e antigens (SEQ. ID. NO:5), and the measles surface protein, as described in US Patent Publication 2010-0322962, the disclosure of which is incorporated by reference in its entirety.

NoV P particles, including NoV P with surface loop viral and bacterial antigens, including RV VP8*, can be presented as a large complex comprising a plurality of dimeric/oligomeric fusion protein, including NoV P proteins and NoV P proteins with surface loop antigens, as described in U.S. Provisional Patent Application 61/670,288, filed Jul. 11, 2012 (Attorney Docket CHM-047P), the disclosure of which is incorporated by reference in its entirety.

The viral protein particles, and in particular, NoV P particles, are comprised in a composition suitable for administration to an egg-laying bird. The composition can include carriers and adjuvants that are well known in the art, and can be an aqueous composition. A carrier can be selected from the group consisting of phosphate, citrate, and other organic acids; antioxidants including ascorbic acid; low molecular weight (less than about 10 residues) polypeptides; proteins, serum albumin, gelatin, immunoglobulins; hydrophilic polymers, polyvinylpyrrolidone; amino acids, glycine, glutamine, asparagine, arginine, lysine; monosaccharides, disaccharides, and other carbohydrates, glucose, mannose, dextrins; chelating agents, EDTA; sugar alcohols, mannitol, sorbitol; salt-forming counterions, sodium; nonionic surfactants, and/or polyethylene glycol (PEG). An adjuvant can be selected from the group consisting of mineral salts, aluminium hydroxide, aluminium or calcium phosphates, gels, oil emulsions, surfactant based formulations, MF59 (microfluidised detergent stabilised oil in water emulsion), QS21 (purified saponin), AS02 (SBAS2, oil-in-water emulsion+monophosphoryl lipid A (MPL)+QS21), Montanide ISA 51, ISA-720 (stabilised water in oil emulsion), Adjuvant 65 (containing peanut oil, mannide monooleate and aluminum monostearate), particulate adjuvants, virosomes (unilamellar liposomal vehicles incorporating influenza haemagglutinin), AS04 (Al salt with MPL), ISCOMS (structured complex of saponins and lipids, polyactide co-glycolide (PLG), microbial derivatives (natural and synthetic), monophosphoryl lipid A (MPL), Detox (MPL+M. Phlei cell wall skeleton), AGP(RC-529 (synthetic acylated monosaccharide)), DC_chol (lipoidal immunostimulators able to self organise into liposomes), OM-174 (lipid A derivative), CpG motifs (synthetic oligonucleotides containing immunostimulatory CpG motifs), modified bacterial toxins, LT and CT, with non-toxic adjuvant effects, Endogenous human immunomodulators, hGM-CSF, hIL-12, Immudaptin (C3d tandem array), inert vehicles, and gold particles.

The birds are immunized with the composition by oral administration or injection. The injection can be an intramuscular injection or a subcutaneous injection. The birds are re-immunized after a period of time comprising a plurality of weeks.

IgY can be extracted from egg yolk by well known means, including water dilution methods that are described in U.S. Pat. No. 5,367,054, and the references and methods described therein, the disclosures of which are incorporated by reference in their entirety.

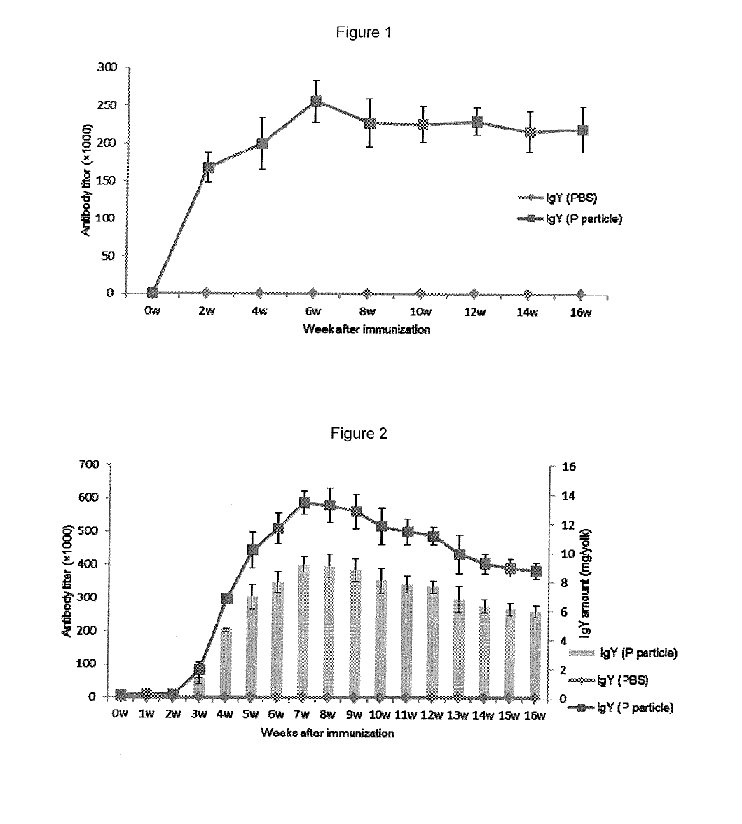

The level of serum and egg yolk anti-NoV and anti-RV IgYs of individual chickens can be measured by ELISA. Steady increases in serum anti-NoV and anti-RV IgY antibody titers are observed after the first immunization and reach a peak at up to the 6^{th} week, and remain high for at least 10 weeks after the peak. By comparison, sera of chickens immunized with the PBS-adjuvant control did not have any reactivity to NoV P particles (FIG. 1a). The anti-NoV and anti-RV antibody titers in the eggs can be detected 2-3 weeks after immunization, and continue increasing, reaching a peak in 6-7 weeks after the first immunization. Chickens continue making high titers of NoV-specific antibodies in their eggs (200,000˜500,000 units), with little variation for at least 13 weeks (about weeks 4-16 after immunization) (FIG. 1b). These antibody titers were similar to the IgG (400,000) from guinea pigs immunized with VLPs of strain VA387. The chicken IgY reacted strongly with the VA387 VLPs (FIG. 1c) and RV VP8* (SEQ. ID. NO:4).

The concentration of total IgY can be determined by SDS-PAGE with standard BSA (bovine serum albumin standard II, Bio-rad) and verified by ELISA using an IgY standard. The concentration of specific anti-NoV IgY was also determined by ELISA. After comparison with the IgY standard curve, the total IgY level in each yolk was estimated to be 186±21 mg. The specific anti-NoV IgY levels can increase from about the third week after the first immunization with an average of 4.7-9.2 mg/yolk from the fourth to sixteenth week, and can accounting for about 2.5-5.0% of the total IgY (FIG. 1b). Analysis of the concentrated egg IgY by SDS-PAGE revealed a major band of ˜68 kDa heavy chain and a minor band of ˜28 kDa light chain and an unknown protein band of ˜40 kDa (FIG. 2a). NoV-specific IgY also reacted with NoV P particles in a Western blot analysis (FIG. 2c).

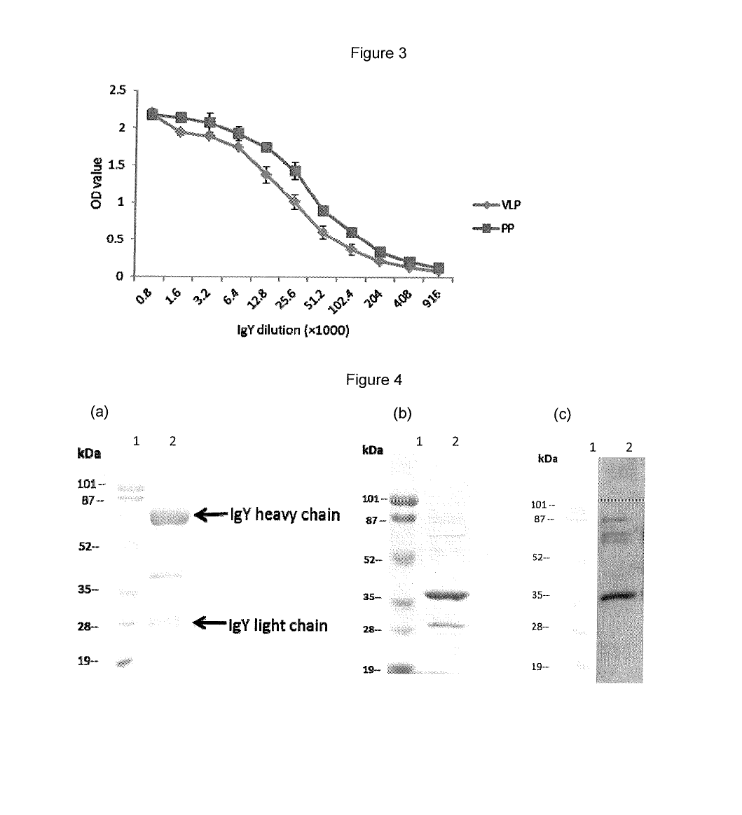

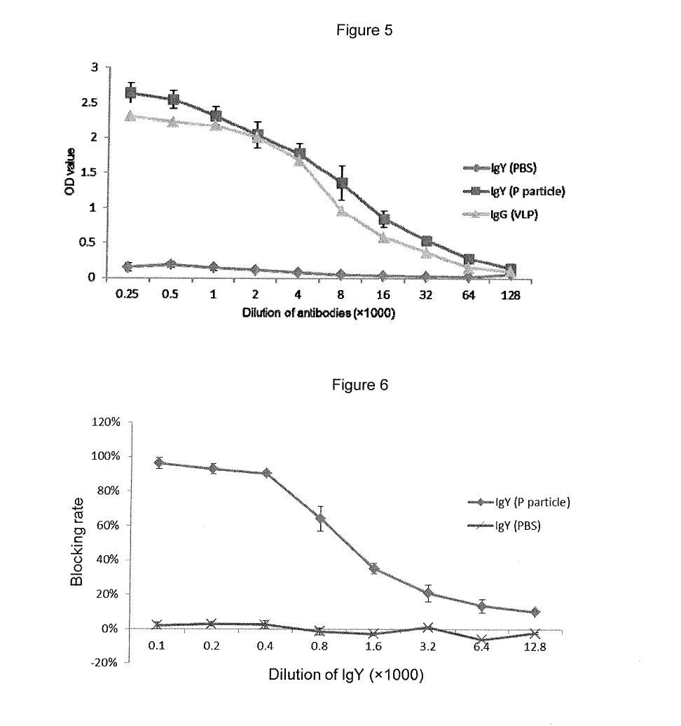

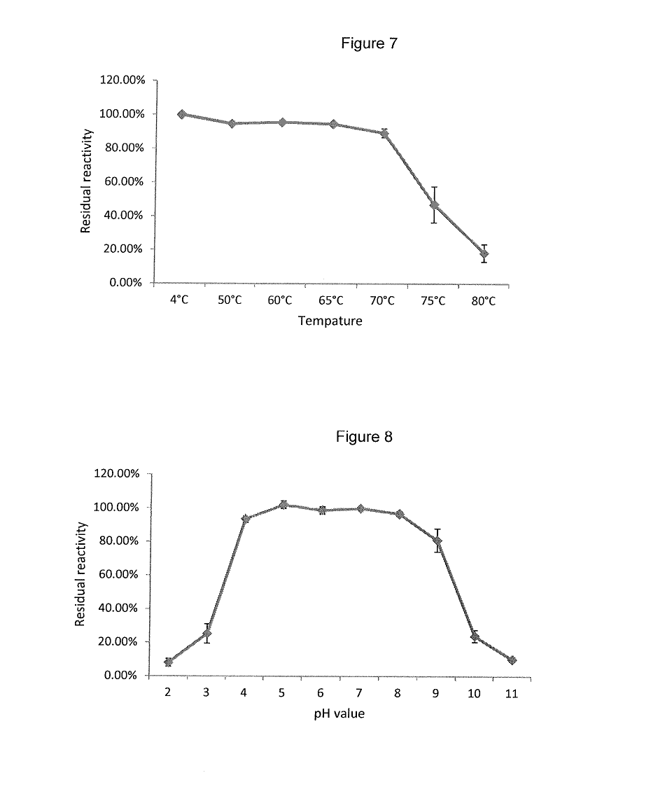

The egg yolk IgY antibodies induced by P particles and their derivatives can be used in an ELISA as a detection antibody to measure NoV/receptor interaction. Partially purified IgY can react strongly with both NoV VLPs or P particles in saliva-based HBGA binding assays, similar to guinea pig IgG after immunization with VLPs of strain VA387 (FIG. 3a). In addition, the egg yolk IgY antibodies are able to block NoV VLP and P particle binding to HBGA receptors (FIG. 3b) with a BT₅₀ (a serum dilution with 50% blocking activity) of about 1:800.

IgY solutions extracted from egg yolk remain are temperature and pH stable, demonstrated by reactive stability to NoV P particles and their derivatives after heat treatment at 70° C. or below for 30 min, as shown by its blocking capability (FIG. 4a), and retained blocking ability after an incubation at pH 4-9 at 37° C. for 3 h (FIG. 4b).

### EXAMPLES

### Immunization of Chickens

Ten, 20-week-old, healthy White Leghorn chickens were provided by the Guangdong bird breeding company (Guangzhou, China) and were randomly divided into two groups. Four chickens (immunization group) were immunized by injecting 50 μg of NoV P particle antigen (SEQ. ID. NO:1) into different spots of the pectoral muscle three times in two week intervals. The first immunization included complete Freund's adjuvant (Sigma, F5881, St Louis, USA), while the second and third boosters were administrated with incomplete Freund's adjuvant (Sigma, F5506, St Louis, USA). The control group (n=6) was injected with PBS plus the corresponding adjuvant. Blood (1 ml) was collected from the wing vein before and after each immunization. Eggs were collected one week before immunization and every day after the first immunization for 16 weeks. The experimental protocol was reviewed and approved by the Ethics Commission for the Use of Animals of School of Public Health and Tropical Medicine, Southern Medical University.

Sera were collected from blood after an overnight incubation at 4° C. and centrifugation at 7000×g for 10 min at 4° C., and stored at −20° C. until use. NoV-specific IgY antibody titers of sera are measured by standard ELISA assays. Briefly, ninety-six well microtiter plates (Dynex Immulon; Dynatech, Franklin, Mass., USA) were coated with 100 μl of purified NoV P particle antigen (SEQ. ID. NO:1, 200 ng/well) and incubated overnight at 4° C. After blocking with 5% nonfat milk, serially diluted chicken sera were added to the antigen-coated wells and incubated at 37° C. for 1 h. After washing, goat anti-chicken IgY-HRP (1:5000) (Santa Cruz Biotechnology, Santa Cruz, Calif., USA) was added. The bound HRP was colorized by adding substrate reagent (BD OptEIA TMB Substrate Reagent Set, BD Biosciences, San Jose, Calif., USA). The signal intensity is measured at 450 nm using a micro-plate reader (DTX 880 Multimode Reader, Beckman Coulter, GmbH, Krefeld, Germany). Pre-immunized chicken sera and chicken sera after immunization with PBS were used as controls. Antigen-specific antibody titers were defined as the end-point dilutions with a cutoff signal intensity of 0.15.

### Isolation and Purification of Yolk IgY

Eggs were stored at 4° C. before IgY extraction. A water dilution method for IgY extraction from egg yolk. Egg yolks were separated from egg whites by egg separators and washed with deionized water. The egg yolk was diluted 10 times with PBS and then the suspension was adjusted to a final pH of 5 with 0.1 N HCl and kept overnight at 4° C. The supernatant containing the IgY was collected after centrifugation (10000×g for 30 min at 4° C.). Solid ammonium sulfate was added to the supernatant to reach 55% saturation and the mixture was kept at 4° C. for 2 h. The precipitate was collected by centrifugation (10000×g for 15 min at 4° C.) and dissolved in 2-4 ml cold PBS, before addition of 50-100 ml (25× volume of PBS) 33% saturated ammonium sulfate (SAS) solution to give a final 31.7% of SAS. The mixture was kept at 4° C. for 2 h. Protein precipitate was collected again by centrifugation (10000×g for 15 min at 4° C.) and was then dissolved in 6-7 ml PBS (pH 7.4). After pasteurization at 60° C. for 30 minutes, the IgY solution was stored at 4° C. The purity of the IgY was determined by sodium dodecyl sulfate-polyacrylamide gel electrophoresis (SDS-PAGE) followed by Coomassie blue staining.

### Determination of NoV-Specific IgY Titers in Egg Yolk

NoV-specific IgY titer in egg yolk was determined by the aforementioned ELISA, in which IgY samples were serially diluted to determine the end-point dilution. To calculate the amount of total IgY and P particle-specific IgY, a standard curve was set up as follows: wells were coated with 100 μl serially diluted pure chicken IgY (Promega, G116A, Madison, Wis., USA) at a concentration from 0.0075 μg/ml to 1 μg/ml. After washing with PBST (PBS containing 0.05% Tween), 100 μl of goat anti-chicken IgY-HRP (1:5000) (Santa Cruz Biotechnology, Calif., USA) were added (37° C. for 1 h). The bound HRP was colorized by substrate reagent (BD OptEIA TMB Substrate Reagent Set; BD Biosciences San Diego, Calif., USA), followed by a reading of the signal intensity at 450 nm (DTX 880 Multimode Reader, Beckman Coulter, GmbH, Krefeld, Germany). The resulting standard curve of absorbance was used to quantify the relative concentration of total IgY and P particle specific IgY from the chickens by coating plates with either P particles or rabbit anti-chicken IgY antibodies (10 μg/ml, Sigma C2288, USA) to capture the total IgY or P particle-specific IgY. Alternatively, to compare the reactivity of IgY induced by NoV P particles to VLPs of strain VA387, plates were coated with either 100 μl VLP (200 ng/well) or P particles in different dilutions, as described above.

### Characterization of IgY by SDS-PAGE and Western Blot Analysis

NoV P particles were separated by conventional SDS-PAGE and visualized with Coomassie Blue stain. For Western analysis, proteins were transferred to nitrocellulose membranes. After blocking with 5% nonfat milk, the membrane was incubated with anti-NoV-specific IgY or non-specific IgY (1:2000) in 1% nonfat milk-PBS at 4° C. overnight. After washing with PBST, the membrane was incubated with goat anti-IgY (Sigma, Steinhein, Germany, 1:5000) at 37° C. for 1 h. The bound HRP was detected by enhanced chemiluminescence (ECL) Western blotting detection reagents (GE Healthcare Life Sciences, Buckinghamshire, England). The ECL signals were captured by Hyper film ECL (GE Healthcare Life Sciences, Buckinghamshire, England).

### HBGA Binding and Blocking Assays

The saliva-based binding and blocking assays were carried out as described Feng and Jiang, 2007, the disclosure of which is incorporated by reference in its entirety. Briefly, boiled human saliva samples with known HBGA phenotypes were diluted 1000-fold and coated on 96-well microtiter plates (Dynex Immulon; Dynatech, Franklin, Mass., USA). After blocking with 5% nonfat milk in PBS, NoV P particles (SEQ. ID. NO:1, 250 ng/ml; VA387, GII.4) were added. The bound P particles were detected using serially diluted IgY (from 1:250-1:128,000), followed by the addition of HRP-conjugated goat anti-chicken IgY (1:5000). Guinea pig antibodies against NoV VLPs were also used in this HBGA binding assay, followed by the addition of HRP-conjugated goat anti-guinea pig IgG (1:5000).

The blocking effects of IgY on P particle binding to saliva samples were measured after pre-incubation of P particles with diluted IgY for 1 hour at 37° C. before addition to the saliva-coated wells. Then a guinea pig anti-VA387 VLP antiserum (1:3333) was added, followed by the addition of HRP-conjugated goat anti-guinea pig IgG (1:5000). The blocking rates were calculated by comparing the optical densities (ODs) measured with and without blocking by the chicken IgYs. The IgYs from chickens immunized with PBS were used as negative controls.

### Reactivity of IgY After Treatments with Various pHs and Temperatures

Egg yolk IgY solution (1 ml, 1:4000, pH 7.4) in a test tube was heated to a given temperature (50 to 80° C.) for up to 30 min. The heated mixtures were then cooled on ice. To determine pH stability, egg yolk IgY solution (1 ml, 1:40, pH 7.4) was diluted with 10 mM phosphate buffer containing 0.15 M NaCl. The pH of the solutions was adjusted with HCl or NaOH to a final pH of 2 to 11. The solution was incubated at 37° C. for 3 h, and then the pH of the solution was neutralized by adding 100×PBS. The activity of the treated IgY was measured by HBGA blocking assay.

The following references are incorporated by reference in their entireties:
Akita, E. M., Nakai, S., 1992. Immunoglobulins from Egg-Yolk—Isolation and Purification. Journal of Food Science 57, 629-634.
Akita, E. M., Nakai, S., 1993. Production and purification of Fab′ fragments from chicken egg yolk immunoglobulin Y (IgY). J Immunol Methods 162, 155-64.
Amaral, J. A., Tino De Franco, M., Carneiro-Sampaio, M. M., Carbonare, S. B., 2002. Anti-enteropathogenic Escherichia coli immunoglobulin Y isolated from eggs laid by immunised Leghorn chickens. Res Vet Sci 72, 229-34.
Cooper, H. M., Paterson, Y., 2009. Production of polyclonal antisera. Curr Protoc Neurosci Chapter 5, Unit 5 5.
Dias da Silva, W., Tambourgi, D. V., 2010. IgY: a promising antibody for use in immunodiagnostic and in immunotherapy. Vet Immunol Immunopathol 135, 173-80.
Feng, X., Jiang, X., 2007. Library screen for inhibitors targeting norovirus binding to histo-blood group antigen receptors. Antimicrob Agents Chemother 51, 324-31.
Glass, R. I., Parashar, U. D., Estes, M. K., 2009. Norovirus gastroenteritis. N Engl J Med 361, 1776-85.
Huang, P., Farkas, T., Marionneau, S., Zhong, W., Ruvoen-Clouet, N., Morrow, A. L., Altaye, M., Pickering, L. K., Newburg, D. S., LePendu, J., Jiang, X., 2003. Noroviruses bind to human ABO, Lewis, and secretor histo-blood group antigens: identification of 4 distinct strain-specific patterns. J Infect Dis 188, 19-31.
Huang, P., Farkas, T., Zhong, W., Tan, M., Thornton, S., Morrow, A. L., Jiang, X., 2005. Norovirus and histo-blood group antigens: demonstration of a wide spectrum of strain specificities and classification of two major binding groups among multiple binding patterns. J Virol 79, 6714-22.
Liou, J. F., Chang, C. W., Tailiu, J. J., Yu, C. K., Lei, H. Y., Chen, L. R., Tai, C., 2010. Passive protection effect of chicken egg yolk immunoglobulins on enterovirus 71 infected mice. Vaccine 28, 8189-96.
Nguyen, H. H., Tumpey, T. M., Park, H. J., Byun, Y. H., Tran, L. D., Nguyen, V. D., Kilgore, P. E., Czerkinsky, C., Katz, J. M., Seong, B. L., Song, J. M., Kim, Y. B., Do, H. T., Nguyen, T., Nguyen, C. V., 2010. Prophylactic and therapeutic efficacy of avian antibodies against influenza virus H5N1 and H1N1 in mice. PLoS One 5, e10152.
Nurminen, K., Blazevic, V., Huhti, L., Rasanen, S., Koho, T., Hytonen, V. P., Vesikari, T., 2011. Prevalence of norovirus GII-4 antibodies in Finnish children. J Med Virol 83, 525-31.
Patel, M. M., Widdowson, M. A., Glass, R. I., Akazawa, K., Vinje, J., Parashar, U. D., 2008. Systematic literature review of role of noroviruses in sporadic gastroenteritis. Emerg Infect Dis 14, 1224-31.
Reeck, A., Kavanagh, O., Estes, M. K., Opekun, A. R., Gilger, M. A., Graham, D. Y., Atmar, R. L., 2010. Serological correlate of protection against norovirus-induced gastroenteritis. J Infect Dis 202, 1212-8.
Schwartz, S., Vergoulidou, M., Schreier, E., Loddenkemper, C., Reinwald, M., Schmidt-Hieber, M., Flegel, W. A., Thiel, E., Schneider, T., 2011. Norovirus gastroenteritis causes severe and lethal complications after chemotherapy and hematopoietic stem cell transplantation. Blood 117, 5850-6.
Tan, M., Fang, P., Chachiyo, T., Xia, M., Huang, P., Fang, Z., Jiang, W., Jiang, X., 2008. Noroviral P particle: structure, function and applications in virus-host interaction. Virology 382, 115-23.
Tan, M., Fang, P.A., Xia, M., Chachiyo, T., Jiang, W., Jiang, X., 2011a. Terminal modifications of norovirus P domain resulted in a new type of subviral particles, the small P particles. Virology 410, 345-52.
Tan, M., Hegde, R. S., Jiang, X., 2004. The P domain of norovirus capsid protein forms dimer and binds to histo-blood group antigen receptors. J Virol 78, 6233-42.
Tan, M., Huang, P., Xia, M., Fang, P. A., Zhong, W., McNeal, M., Wei, C., Jiang, W., Jiang, X., 2011b. Norovirus P particle, a novel platform for vaccine development and antibody production. J Virol 85, 753-64.
Tan, M., Jiang, X., 2005. The p domain of norovirus capsid protein forms a subviral particle that binds to histo-blood group antigen receptors. J Virol 79, 14017-30.
Tan, M., Jiang, X., 2010. Norovirus gastroenteritis, carbohydrate receptors, and animal models. PLoS Pathog 6.
Vega, C., Bok, M., Chacana, P., Saif, L., Fernandez, F., Parreno, V., 2011. Egg yolk IgY: protection against rotavirus induced diarrhea and modulatory effect on the systemic and mucosal antibody responses in newborn calves. Vet Immunol Immunopathol 142, 156-69.
Xu, Y., Li, X., Jin, L., Zhen, Y., Lu, Y., Li, S., You, J., Wang, L., 2011. Application of chicken egg yolk immunoglobulins in the control of terrestrial and aquatic animal diseases: A review. Biotechnol Adv.

We claim:

1. A method for producing NoV-specific IgY or IgY against other bacterial or viral antigens, in the egg yolks of birds in large amounts and with high titer, by immunizing the birds with NoV P particles or a P particles carrying other, variable surface bacterial or viral antigens.

2. A method for passive immunization of a mammal against NoV or other bacterial or viral antigen infection, comprising administering to a mammal having a compromised or weakened immunity system, at least one dose of a composition comprising NoV-specific IgYs or IgY against other bacterial or viral antigens in an amount sufficient to treat, ameliorate, inhibit or prevent the NoV or other bacterial or viral infection.

3. A method for generating IgY antibodies, comprising the step of immunizing each of a plurality of egg-laying birds with a composition comprising an antigen, the antigen comprising a viral protein particle, thereby generating IgY antibodies.

4. The method according to claim 3 wherein the viral protein particle comprises a NoV P particle (SEQ. ID. NO:1).

5. The method according to claim 3 wherein the viral protein particle comprises a NoV P particle carrying at least one surface antigen.

6. The method according to claim 5 wherein the at least one surface antigen is selected from the group consisting of a bacterial antigen and a viral antigen.

7. The method according to claim 6 wherein at least one surface antigen is selected from the group consisting of the rotavirus spike protein VP8* antigen (SEQ. ID. NO: 5), influenza virus M2e antigen (SEQ. ID. NO:6), and the measles surface protein.

8. The method according to claim 6 wherein the at least one surface antigen is a viral antigen.

9. The method according to claim 8 wherein the viral antigen is the rotavirus spike protein VP8* antigen (SEQ. ID. NO:5).

10. The method according to claim 8 wherein the viral antigen is the influenza virus M2e antigen (SEQ. ID. NO:6).

11. The method according to claim 3 comprising the further step of extracting the IgY antibodies from eggs laid by the birds.

12. The method according to claim 11 comprising the further step of purifying the IgY antibodies.

13. The method according to claim 3 wherein the composition comprises a plurality of different viral protein particles.

14. The method according to claim 3, wherein the plurality of egg-laying birds comprises a plurality of egg-laying birds of different species.

15. The method according to claim 3, wherein the birds are immunized by oral administration or injection of the composition.

16. The method according to claim 15, wherein the birds are re-immunized after a period of time comprising a plurality of weeks.

17. The method according to claim 15, wherein the injection is an intramuscular injection or a subcutaneous injection.


SEQUENCE LISTING (5 sequences)

SEQ ID NO: 1 - protein, 318 aa, Artificial Sequence
  NoV P particle of VA387
SRTKPFTVPILTVEEMSNSRFPIPLEKLYTGPSSAFVVQPQNGRCTTDGVLLGTTQLSAVNICTFRGDVTHIAGSHDYIMNLASQNWNNYDPTEEIPAPLGTPDFVGKIQGMLTQTTREDGSTRAHKATVSTGSVHFTPKLGSVQYTTDTNNDLQTGQNTKFTPVGVIQDGNNHQNEPQQWVLPNYSGRTGHNVHLAPAVAPTFPGEQLLFFRSTMPGCSGYPNMNLDCLLPQEWVQHFYQEAAPAQSDVALLRFVNPDTGRVLFECKLHKSGYVTVAHTGPHDLVIPPNGYFRFDSWVNQFYTLAPMGNGAGRRRAL

SEQ ID NO: 2 - protein, 538 aa, Artificial Sequence
  GST-NoV P-
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRSRTKPFTVPILTVEEMSNSRFPIPLEKLYTGPSSAFVVQPQNGRCTTDGVLLGTTQLSAVNICTFRGDVTHIAGSHDYIMNLASQNWNNYDPTEEIPAPLGTPDFVGKIQGMLTQTTREDGSTRAHKATVSTGSVHFTPKLGSVQYTTDTNNDLQTGQNTKFTPVGVIQDGNNHQNEPQQWVLPNYSGRTGHNVHLAPAVAPTFPGEQLLFFRSTMPGCSGYPNMNLDCLLPQEWVQHFYQEAAPAQSDVALLRFVNPDTGRVLFECKLHKSGYVTVAHTGPHDLVIPPNGYFRFDSWVNQFYTLAPMGNGAGR

SEQ ID NO: 3 - protein, 224 aa, Artificial Sequence
  GST
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPR

SEQ ID NO: 4 - protein, 159 aa, Artificial Sequence
  Rotavirus VP8*
LDGPYQPTTFTPPTDYWILINSNTNGVVYESTNNSDFWTAVIAVEPHVNPVDRQYNVFGENKQFNVRNDSDKWKFLEMFRGSSQNDFYNRRTLTSDTRLVGILKYGGRIWTFHGETPRATTDSSNTANLNGISITIHSEFYIIPRSQESKCNEYINNGL

SEQ ID NO: 5 - protein, 23 aa, Artificial Sequence
  influenza virus M2e antigen
SLLTEVETPIRNEWGCRCNDSSD